(19) United States
(12) Patent Application Publication
(10) Pub. No.: US 2024/0215873 A1
(43) Pub. Date: Jul. 4, 2024

(54) Aptamer Sensors with Temperature Correction

(71) Applicant: University of Cincinnati, Cincinnati, OH (US)

(72) Inventors: Jason Heikenfeld, Cincinnati, OH (US); Israel Belmonte, Cincinnati, OH (US); Ryan Jeffrey White, Cincinnati, OH (US)

(73) Assignee: University of Cincinnati, Cincinnati, OH (US)

(21) Appl. No.: 18/684,114

(22) PCT Filed: Aug. 22, 2022
(86) PCT No.: PCT/US22/41118

§ 371 Date: Feb. 15, 2024

Related U.S. Application Data
(60) Provisional application No. 63/235,349, filed on Aug. 20, 2021.

(51) Int. Cl. A61B 5/145; A61B 5/1459; A61B 5/1473; G01N 27/27; G01N 27/327; G01N 33/53; G01N 33/543

(52) U.S. Cl. CPC A61B 5/14546; A61B 5/1459; A61B 5/1473; G01N 27/27; G01N 27/3276; G01N 27/3277; G01N 33/5308; G01N 33/5438; A61B 2560/0252

(57) ABSTRACT

A device for measuring one or more analytes in a sample fluid is provided. The device includes at least one aptamer sensor comprising a plurality of aptamers. The sensor provides a measurement of at least one of the analytes. In addition, the measurement is affected by changes in temperature (“a temperature-dependent response”). The device also includes a means to establish fluid communication between the at least one aptamer sensor and the sample fluid. Further, the device includes at least one temperature sensor and a means for correcting a measurement error due to the temperature-dependent response.

### CROSS-REFERENCE TO RELATED APPLICATIONS

This application is a continuation of PCT Application No. PCT/US22/41118 filed Aug. 22, 2022, which claims benefit of the filing date of U.S. Provisional Application No. 63/235,349 filed Aug. 20, 2021, the disclosures of which are incorporated by reference herein in their entireties.

### TECHNICAL FIELD

The present invention relates to the use of electrochemical, aptamer-based (EAB) sensors.

### BACKGROUND OF THE INVENTION

Electrochemical aptamer sensors consist of an aptamer sequence that specifically binds to an analyte of interest, attached to an electrode, and the aptamer having an attached redox active molecule (redox tag) which can transfer electrical charge to or from the electrode. When an analyte binds to the aptamer, the aptamer changes shape, bringing the redox tag closer or further, on average, from the electrode, resulting in a measurable change in electrical current that can be translated to a measure of concentration of the analyte. A major unresolved challenge for aptamers is temperature correction for point of care, wearable, and implantable applications. Aptamer-based sensing has largely been a laboratory project limited to environmentally controlled testing even for simple in-vitro (beaker) tests and for animal tests. In real-world application of aptamer-based sensors temperature may be a confounding factor to measurement accuracy.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sensing technology into proximity with sample fluids containing at least one analyte of interest to be measured.

Embodiments of the disclosed invention are directed to aptamer sensors with temperature correction.

And so, one aspect of the present invention is directed to a device for measuring one or more analytes in a sample fluid. The device includes at least one aptamer sensor comprising a plurality of aptamers. The sensor provides a measurement of at least one of the analytes. In addition, the measurement is affected by changes in temperature (“a temperature-dependent response”). The device also includes a means to establish fluid communication between the at least one aptamer sensor and the sample fluid. Further, the device includes at least one temperature sensor and a means for correcting a measurement error due to the temperature-dependent response. In one embodiment, the sample fluid is selected from the group consisting of interstitial fluid, blood and combinations thereof. In another embodiment, the device also includes a reader component with at least one temperature sensor.

In one embodiment, the aptamer sensor is capable of being placed subcutaneously. In another embodiment, the temperature sensor is capable of being placed subcutaneously. In one embodiment, the temperature sensor is capable of being placed outside the skin. In another embodiment, there is a first temperature sensor that is capable of being placed subcutaneously and there is a second temperature sensor that capable of being placed outside the skin.

In one embodiment, the temperature sensor is located no more than about 3 mm from at least one electrochemical aptamer sensor. In another embodiment, the temperature sensor is located no more than about 1 mm from at least one electrochemical aptamer sensor. In one embodiment, the temperature sensor is located no more than about 300 μm from at least one electrochemical aptamer sensor. In another embodiment, the temperature sensor is located no more than about 100 μm from at least one electrochemical aptamer sensor. In one embodiment, the device also includes thermal insulation on the device such that the temperature sensor provides adequate prediction of the temperature of at least one electrochemical aptamer sensor.

In another embodiment, the device of the present invention also includes at least one component which is capable of containing a record or prediction of a temperature dependence of the aptamer sensor. In one embodiment, the aptamer sensor is an electrochemical aptamer sensor with a redox tag attached to each of the plurality of aptamers and said record or prediction additionally comprises a frequency response of the aptamer. In another embodiment, the aptamer sensor is an electrochemical aptamer sensor with a redox tag attached to each of the plurality of aptamers and said record or prediction additionally comprises an analyte concentration response of the aptamer.

In one embodiment, the aptamer sensor is an electrochemical aptamer sensor with a redox tag attached to each of the plurality of aptamers and said record or prediction additionally comprises a change in sensor response over multiple hours of use of the sensor. In another embodiment, the aptamer sensor is optical and carries at least one fluorescent tag on each of the plurality of aptamers.

Another aspect of the present invention is directed to a method for measuring one or more analytes in a sample fluid. The method involves first exposing a sample fluid comprising one or more analytes to at least one aptamer sensor. The aptamer sensor includes a means to establish fluid communication between the at least one aptamer sensor and the sample fluid. The aptamer sensor also includes at least one temperature sensor. The method further involves detecting a concentration of said one or more analytes in the sample using the sensor. The temperature sensor takes at least one measurement and the measurement is a referenced record or prediction of a temperature dependence of the aptamer sensor that predicts change in aptamer sensor response to change in temperature. Also, the signal measured by the aptamer sensor is adjusted by using the measurement of the temperature sensor and the record or prediction such that the signal from the aptamer sensor accurately represents analyte concentration.

In one embodiment, the sample fluid is selected from the group consisting of interstitial fluid, blood and combinations thereof. In another embodiment, the sensor further includes a reader component with at least one temperature sensor, and further, wherein the aptamer sensor is disposable after use.

In one embodiment, the aptamer sensor is placed subcutaneously. In another embodiment, the temperature sensor is placed subcutaneously. In one embodiment, the temperature sensor is placed outside the skin. In another embodiment, there is a first temperature sensor is placed subcutaneously and a second temperature sensor is placed outside the skin.

In one embodiment, the temperature sensor is located no more than about 3 mm from at least one electrochemical aptamer sensor. In another embodiment, the temperature sensor is located no more than about 1 mm from at least one electrochemical aptamer sensor. In one embodiment, the temperature sensor is located no more than about 300 μm from at least one electrochemical aptamer sensor. In another embodiment, the temperature sensor is located no more than about 100 μm from at least one electrochemical aptamer sensor.

In one embodiment, thermal insulation is further included on the device such that the temperature sensor provides adequate prediction of the temperature of at least one electrochemical aptamer sensor. In another embodiment, the aptamer sensor is an electrochemical aptamer sensor with a redox tag attached to each of the plurality of aptamers and said record or prediction additionally contains a frequency response of the aptamer. In one embodiment, the aptamer sensor is an electrochemical aptamer sensor with a redox tag attached to each of the plurality of aptamers and said record or prediction additionally contains an analyte concentration response of the aptamer.

In another embodiment, the aptamer sensor is an electrochemical aptamer sensor with a redox tag attached to each of the plurality of aptamers and said record or prediction additionally contains a change in sensor response over multiple hours of use of the sensor. In one embodiment, the aptamer sensor is optical and carries at least one fluorescent tag on each of the plurality of aptamers. In another embodiment, the measurement of the temperature sensor is a direct measure of the temperature at the aptamer sensor. In one embodiment, the measurement of the temperature sensor is an indirect measure of the temperature at the aptamer sensor. In another embodiment, the electrochemical aptamer sensor is attached to a user by placing at least a portion of the sensor in the user's skin.

In one embodiment, the temperature sensor is inserted at least 100 μm into a user's skin. In another embodiment, at least two temperature sensors are placed at different depths into or distances from a user's skin to measure a temperature gradient that predicts temperature of the aptamer sensor. In one embodiment, the aptamer sensor comprises thermal insulation such that the temperature sensor provides adequate prediction of the temperature of the aptamer sensor.

### BRIEF DESCRIPTION OF THE DRAWINGS

The objects and advantages of the disclosed invention will be further appreciated in light of the following detailed descriptions and drawings in which:

FIG. 1 is a cross-sectional view of a device according to an embodiment of the disclosed invention.

FIG. 2 is a cross-sectional view of a device according to an embodiment of the disclosed invention.

FIG. 3 is a cross-sectional view of a device according to an embodiment of the disclosed invention.

FIG. 4 is a schematic of an E-AB Sensor.

FIG. 5A is a graph showing square wave voltammograms demonstrating sensor equilibration, response to their target analytes and regeneration back to baseline after rinsing with water for the destabilized ATP E-AB sensors.

FIG. 5B is a graph showing square wave voltammograms demonstrating sensor equilibration, response to their target analytes and regeneration back to baseline after rinsing with water for the parent tobramycin E-AB sensors.

FIG. 5C is a graph showing square wave voltammograms demonstrating sensor equilibration, response to their target analytes and regeneration back to baseline after rinsing with water for the cocaine E-AB sensors.

FIG. 6A is a graph showing square wave voltammograms of the equilibrations of the Dest. ATP E-AB sensor at several temperatures including room temperature (RT).

FIG. 6B is a graph showing square wave voltammograms of the equilibrations of the Parent Tobramycin E-AB sensor at several temperatures including room temperature (RT).

FIG. 6C is a graph showing square wave voltammograms of the equilibrations of the Cocaine E-AB sensor at several temperatures including room temperature (RT).

FIG. 7A is a graph showing frequency sweeps performed using the Destabilized ATP E-AB sensors at 1° C. (bolded), room temperature (22° C., unbolded) and 37° C. (triangles).

FIG. 7B is a graph showing frequency sweeps performed using the Parent Tobramycin E-AB sensors at 1° C. (unbolded), room temperature (22° C., bolded) and 37° C. (dashed lines).

FIG. 7C is a graph showing frequency sweeps performed using the Cocaine E-AB sensors at 1° C. (unbolded), room temperature (22° C., bolded) and 37° C. (dashed lines).

FIG. 8A is a graph showing calibration curves performed using the Parent Tobramycin E-AB sensors at 1° C. (unbolded), room temperature (bolded) and 37° C. (dashed lines).

FIG. 8B is a graph showing calibration curves performed using the Destabilized ATP E-AB sensors at 1° C. (unbolded), room temperature (bolded) and 37° C. (dashed lines).

FIG. 8C is a graph showing calibration curves performed using the Cocaine E-AB sensors at 1° C. (unbolded), room temperature (bolded) and 37° C. (dashed lines).

FIG. 9 is a 3-D plot of the destabilized ATP E-AB sensors depicting the increase in percent signal change as a function of time (min) and increasing temperature (° C.) without any target analyte added.

FIG. 10A is an illustration showing how the temperature dependence is related to the structural motif of the specific aptamer. This illustration shows an unfolded aptamer-based sensor.

FIG. 10B is an illustration showing how the temperature dependence is related to the structural motif of the specific aptamer. This illustration shows a target-bound state that is folded to keep the redox marker in proximity to the electrode.

FIG. 10C is an illustration showing how the temperature dependence is related to the structural motif of the specific aptamer. This illustration shows another target-bound state that is folded to keep the redox marker in proximity to the electrode.

### DEFINITIONS

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the term “aptamer” means a molecule that undergoes a conformation change as an analyte binds to the molecule, and which satisfies the general operating principles of the sensing method as described herein. Such molecules are, e.g., natural or modified DNA, RNA, or XNA oligonucleotide sequences, spiegelmers, peptide aptamers, and affimers. Modifications may include substituting unnatural nucleic acid bases for natural bases within the aptamer sequence, replacing natural sequences with unnatural sequences, or other suitable modifications that improve sensor function. Aptamers can be optical or electrochemically detectable in nature using attached fluorescent and optical question tags as used in molecular beacons, or for example redox molecule tagged as is used in electrochemical aptamer-based sensors. Aptamers can function as a single nucleotide strand or as two or more strands whose binding to each other is changed in the presence of an analyte to be measured.

The devices and methods described herein encompass the use of sensors. A “sensor”, as used herein, is a device that is capable of measuring the concentration of a target analyte in solution. As used herein, an “analyte” may be any inorganic or organic molecule, for example: a small molecule drug, a metabolite, a hormone, a peptide, a protein, a carbohydrate, a nucleic acid, or any other composition of matter. The target analyte may comprise a drug. The drug may be of any type, for example, including drugs for the treatment of cardiac system, the treatment of the central nervous system, that modulate the immune system, that modulate the endocrine system, an antibiotic agent, a chemotherapeutic drug, or an illicit drug. The target analyte may comprise a naturally-occurring factor, for example a hormone, metabolite, growth factor, neurotransmitter, etc. The target analyte may comprise any other species of interest, for example, species such as pathogens (including pathogen induced or derived factors), nutrients, and pollutants, etc.

As used herein, the term “continuous sensing” simply means the device records a plurality of readings over time.

As used herein, the term “temperature measurement component” or “temperature sensor” means any component such as infrared detector that provides contact or non-contact measurement, thermocouples or thermistors, environmental temperature measurement, or other measurements, devices, or components, which provides a measure of temperature that can be used to improve aptamer sensor measurement accuracy.

As used herein, the term “analyte” means any solute in a solution or fluid which can be measured using a sensor. Analytes can be small molecules, proteins, peptides, electrolytes, acids, bases, antibodies, molecules with small molecules bound to them, DNA, RNA, drugs, chemicals, pollutants, or other solutes in a solution or fluid.

As used herein, the term “sample fluid” means any solution or fluid that contains at least one analyte to be measured.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Certain embodiments of the disclosed invention show sensors as simple individual elements. It is understood that many sensors require two or more electrodes, reference electrodes, or additional supporting technology or features which are not captured in the description herein. Sensors measure a characteristic of an analyte. Sensors are preferably electrical in nature, but may also include optical, chemical, mechanical, or other known biosensing mechanisms. Sensors can be in duplicate, triplicate, or more, to provide improved data and readings. Sensors may provide continuous or discrete data and/or readings. Certain embodiments of the disclosed invention show sub-components of what would be sensing devices with more sub-components needed for use of the device in various applications, which are known (e.g., a battery, antenna, adhesive), and for purposes of brevity and focus on inventive aspects, such components may not be explicitly shown in the diagrams or described in the embodiments of the disclosed invention.

As will be detailed in the examples section, the binding affinities, electron transfer rates, and response curves of aptamer sensors can have a strong and unique temperature dependence compared to other biosensors. This temperature dependence could affect the accuracy of a measurement of an analyte. For example, a point of care testing device that operates similar to a glucose-test-strip reader could be used outside in hot or cold temperatures (ranging from as much as >45° C. in a hot car to <0° C. outside in winter). For example, a wearable device with a sensor implanted into the skin using a small needle-like sensor in the dermis, would be sensitive to changes in both body core temperature, skin surface temperature (radiant heat, sunlight, cool blowing air, water), or ambient temperature. Likewise, most electrochemical and optical implantable devices for biosensing will be inserted under the skin and near the skin surface and can suffer temperature changes as well. Skin temperatures can vary widely with depth into the skin and due to body core and external influences. For example, temperature can range from being >40° C. at the skin surface to 37° C. in the body core, or <30° C. or even <10° C. at the skin surface with 37° C. in the body core, with the temperature drop occurring rapidly over the first 2-10 mm of skin depth. This skin depth is the same depth at which many indwelling needle-based sensors interface with the dermis, hence their operating temperature may not be determined by the external or internal body temperature measurements and could be in between either extreme. Furthermore, a sensor which is ex-vivo on the skin (outside the body) would have a temperature that is in between body temperature and externally induced temperatures. These temperatures will vary with external factors (radiant heat, air flow, evaporation from body surface, ambient temperature, core temperature, clothing, etc.). Therefore, techniques are needed to accurately predict the temperature of the aptamer sensor while it is measuring an analyte.

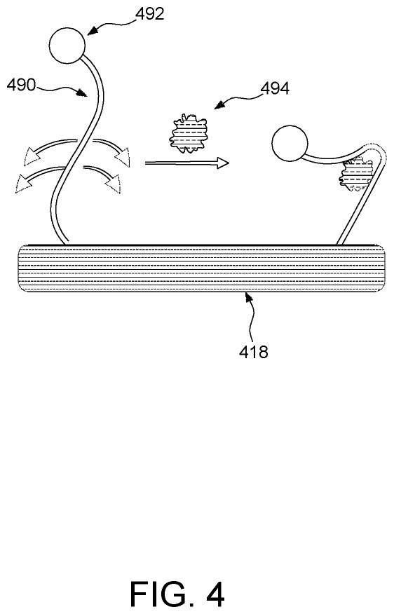

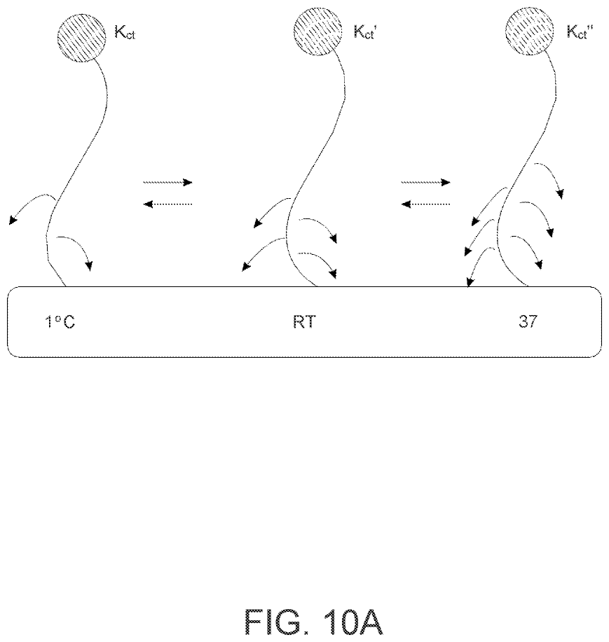

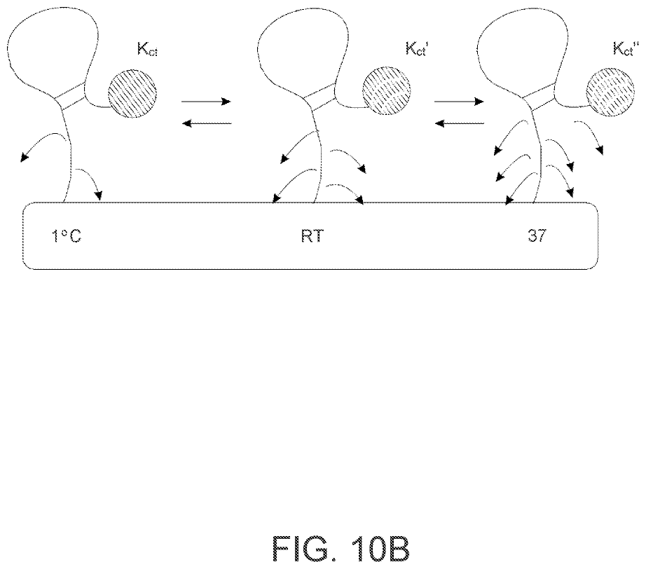

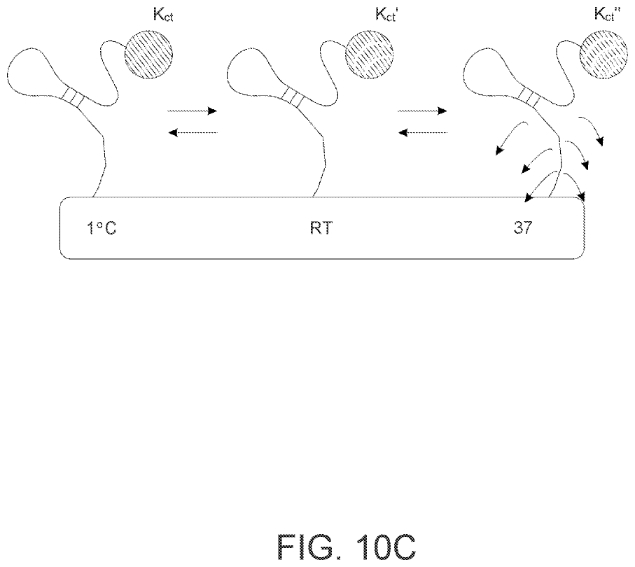

The prediction of the temperature dependence of an aptamer sensor can be generalized or predicted in theory, but in practice has a strong dependence on the actual aptamer structural motif itself and other aspects of the sensor that may affect sensor read-out (such as temperature dependence of basic redox electron transfer to the electrode). Temperature correction for folding-based, aptamer-based sensors needs to take into account the structure of the aptamer in both the target-free state and target-bound state (e.g FIG. 4, FIGS. 10A-C). More specifically, the target free state yields a reproducible current at a given temperature. A target-bound state that is folded to keep the redox marker in proximity to the electrode (FIG. 10B) may yield a higher peak current than an unstructured probe that has more freedom to explore space. As temperature increases, the signal readout (current) increases monotonically some aptamer probes. The caveat is that the change in current varies based on the aptamer structure. The rate of signal change (reported as current density vs. temperature) is not constant throughout the change in temperature presumably due to structural changes in the aptamer (e.g., melting of tertiary/secondary structures). The same observation is made when comparing temperature effects on current for the target-bound states. As such, each sensor needs individual calibration dependent on the structural motifs individualized to each nucleic acid sequence.

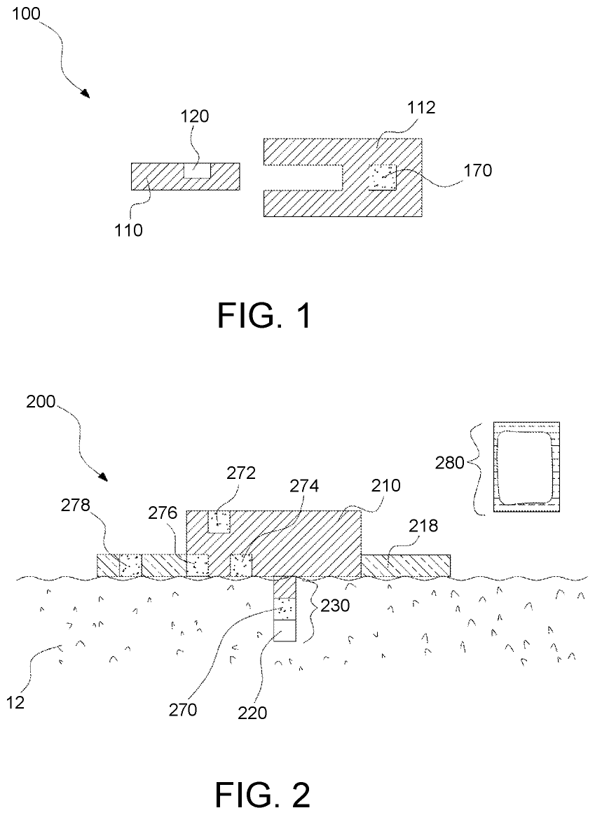

With reference to FIG. 1, in an embodiment of the disclosed invention, a point of care or other type of blood or urine or other biofluid test strip device 100 consists of a test strip 110 made out of plastic with at least one aptamer sensor 120 such as an electrochemical aptamer sensor for tobramycin, vancomycin, cortisol, or other analyte. In one embodiment, the test strip sensor has three electrode fan-outs (counter, working, and reference electrode) that connect to an electrochemical reader 112 with at least one temperature sensor 170. In another embodiment, the test strip uses optical based aptamers such as molecular beacons and the reader 112 is a fluorescent reader. In either case, aptamer binding kinetics have a temperature dependence (see Examples section) that could require temperature correction if the reader and test strip are tested in a warm or cool environment. Temperature sensor 170 may be a simple thermocouple. In one embodiment, the test strip 110 is inserted, and temperature sensor 170 has a look-up table or formula that is used to correct the temperature dependence of the electrochemical measurement of the test strip 110 for the current temperature during which the measurement is made.

With reference to FIG. 2, where like numerals refer to like features, in an embodiment of the disclosed invention, an indwelling or microneedle wearable sensor device 200 with a plastic housing 210 and skin adhesive 218 has an aptamer sensor 220 on an indwelling needle 230 that is inserted subcutaneously at least 100 μm into the skin 12. The device 200 further carries at least one temperature sensor 270, 272, 274, 276, 278. The measurement performed by the aptamer sensor 220 is corrected for temperature variations, because the sensor 220 is not inserted deep enough into the body (cm's or more) to fully avoid temperature variation influences as previously described above. For example, temperature sensor 270 could measure the temperature in the skin, and ideally measure the temperature inside the skin within, in one embodiment, at least 3 mm of the aptamer sensor 220. In another embodiment, the temperature sensor 270 can measure the temperature in the skin within 1 mm of the aptamer sensor 220. In yet another embodiment, the temperature sensor 270 can measure the temperature in the skin within 300 μm of the aptamer sensor 220. In still another embodiment, the temperature sensor 270 can measure the temperature in the skin within 100 μm of the aptamer sensor 220. This can be referred to as a direct measurement of the temperature of the sensor 220. Alternately, temperature can be indirectly measured and determined by one or more other techniques with the sensor itself. For example, a reference electrode has a temperature dependence that can be measured to predict changes in temperature, such as 1 mV per degree Celsius change in temperature, or the peak frequency for scanning wave voltammetry measurement of an electrochemical aptamer sensor will shift with temperature, as diffusion coefficient is temperature dependent, or the background current during a measurement will increase with temperature. One or more of these factors can individually or together provide a temperature measurement through the sensor 220 itself and that temperature measurement is used to correct for a temperature induced shift signal response of the aptamer when it binds to the analyte. Alternately, temperature can be indirectly measured and determined by one or more other temperature sensors 270, 272, 274, 276, 278. For example, sensors 274 and 270, or sensors 272 and 274 can measure a temperature gradient outward from the body surface as well as the temperature gradient used to predict the temperature at the sensor 220, so long as the sensor is at a known depth into the body and body core temperature remains stable or is measured directly. This is achievable because temperature gradients across the body and a device on the body can be easily characterized across a wide spectrum of hot and cold environments. This data can be used to then predict temperature at the sensor once a local temperature gradient is measured using two or more temperature sensors 270, 272, 274, 276, 278. Alternatively, body core temperature can be measured, using for example an optical/infrared temperature measurement such as sensor 274 and sensor 272 measuring external temperature, again being used to predict the temperature of the aptamer sensor 220 so long as it is at the correct depth. Additional temperature data such as ambient temperature can be integrated with the device or software that works with the data from the device 200, for example being measured and or calculated using an additional device 280 such as a smartphone. Alternately, a device 200 could be adequately thermally insulated from ambient temperature such that a single temperature measurement beneath the device and near skin such as sensors 274 or 270 would provide an adequately accurate predictor of temperature at the sensor 220.

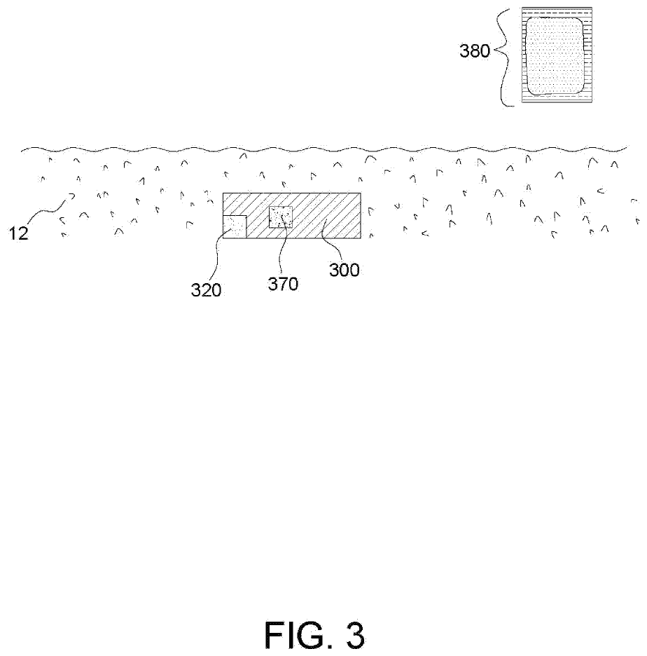

With reference to FIG. 3, where like numerals refer to like features, in an embodiment of the disclosed invention, an implanted device 300, implanted in skin 12, has an aptamer sensor 320 and temperature sensor 370. For an implanted device, directly measuring the local temperature is the most practical approach and more easily performed without additional cost compared to a wearable device. However, a wearable device can also use temperature measurement techniques as previously shown and may include, for example, a wearable electronic reader device 380 that is placed near or on the skin to receive data from implanted device 300, or for example from a smart-watch with temperature sensing that is connected to the same smart phone that is connected to the implanted device 300 (not shown).

With reference to embodiments of the present invention a sensor in the body could also include a local heater with the sensor or with the wearable that would always raise the temperature of the sensor to a predictable value (such as 33 Celsius, or 37 Celsius, for example), but such technique would require a temperature sensor to operation and therefore require one or more elements as taught in for measuring temperature at or near the aptamer sensor.

With reference to embodiments of the present invention, aptamer sensors have the potential to be employed as long-term monitoring devices, point-of-care (POC) along with other opportunities. Their performance is dependent on parameters such as buffer solutions (electrolyte concentration and pH), surface coverage on the conductive substrate which is commonly gold and the frequency at which they are interrogated, and as taught herein can be dependent on temperature. In the Examples, temperature was accounted for as another parameter to consider when tailoring sensor performance to a specific application. FIG. 4 illustrates an aptamer 490 on electrode 418 with redox tag 492 such as methylene blue that responds to analyte 494 binding and the thermal motion of the aptamer and other factors impacting aptamer sensor signal are dependent on temperature. Electrode 418 may also be coated with a blocking layer such as a monolayer alkythiolate molecules (not shown) which may also have a temperature dependence for electrode transfer between the redox tag 492 and the electrode 418.

With reference to embodiments of the present invention, in order to determine the temperature dependence of an aptamer sensor one or more methods may be employed. Several non-limiting examples are provided. Sensors could be calibrated against a plurality of temperatures using known concentrations of analytes, known measurement techniques (such as frequency response with square wave voltammetry) and known temperatures used during that calibration (for example, calibrated in temperature-controlled serum baths). The sensors response to at least one concentration of analyte could be, at the time of sensor development or sensor manufacturing be measured at two or more temperatures, and more preferably over a full temperature sweep of possible operating temperatures. For a given temperature or for a plurality of temperatures, the sensor response to change in analyte concentration, to measurement parameter (such as frequency) or other temperature dependent aspects of the sensor can be measured at the time of sensor development or sensor manufacturing. These measurements can then be placed into software via look-up tables, or approximated predictive equations or algorithms, or into analog electronics, or other suitable techniques, all of which correct for temperature dependence by transforming a raw sensor signal into an accurate measurement of analyte concentration. The correction can be complex or simple, for example if simple using a simple equation that predicts a change in sensor signal at 10% maximum measurable analyte concentration that increases by 2% per degree Celsius from 27 to 30 Celsius and then by 3% per degree Celsius from 30 to 36 Celsius. The correction can be complex or simple, for example if complex using a full lookup table for each 0.1 degree Celsius change in temperature response over 30 to 40 degrees Celsius over the full range of analyte concentrations and over each 6 hours of operations since temperature dependence can change over time as factors such as sensor fouling impact temperature dependence by restricting freedom of movement of the aptamer.

### EXAMPLES

### Example 1

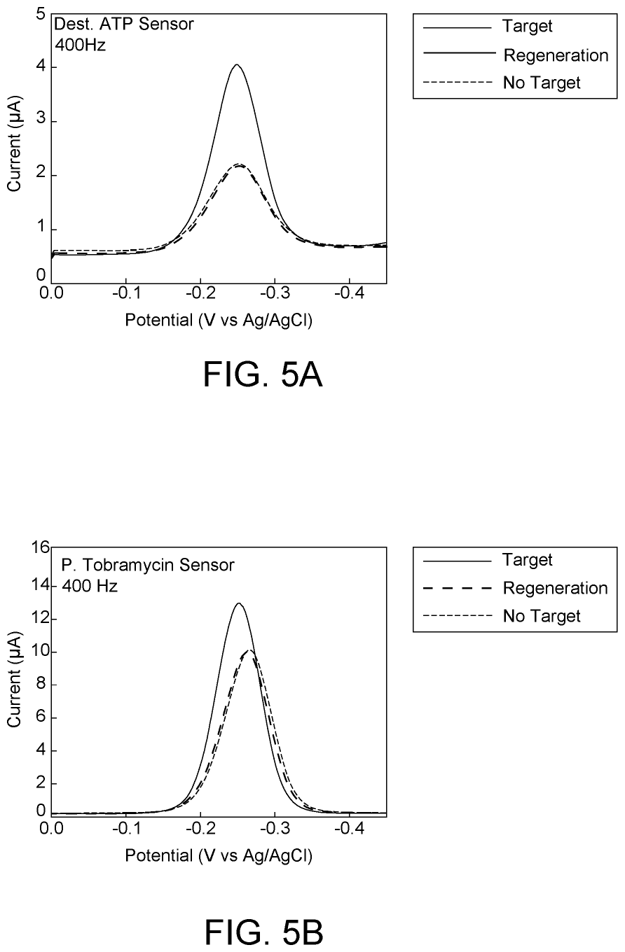

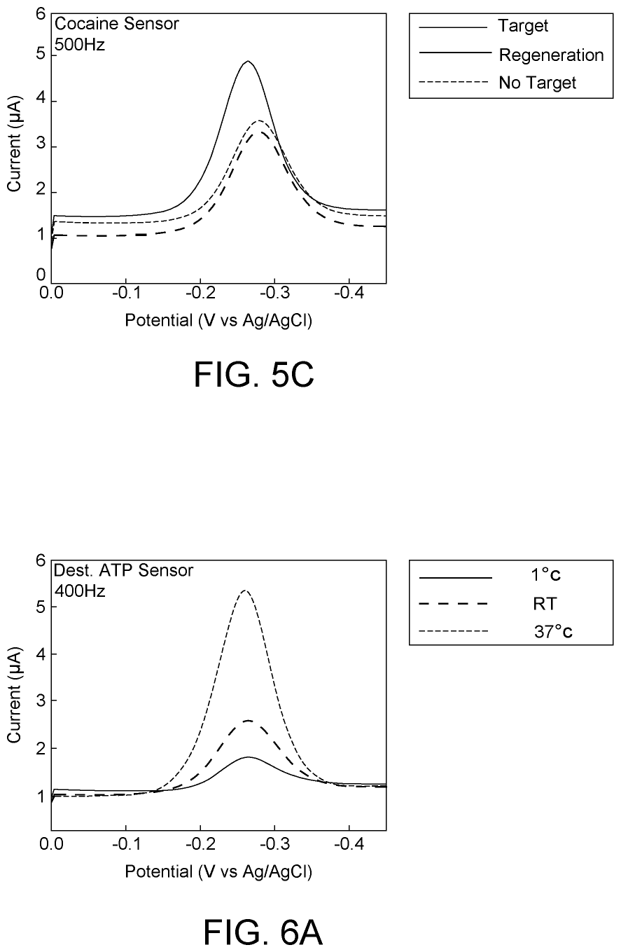

FIGS. 5A-5C are a series of square wave voltammograms demonstrating sensor equilibration (“no target”), response to their target analytes (“target”) and regeneration back to baseline after rinsing with water (“regeneration”) all at room temperature of the destabilized ATP, parent tobramycin and cocaine E-AB sensors, respectively. The sensors were all interrogated in 20 mM tris buffer (20 mM trizma base, 100 mM NaCl and 5 mM MgCl2). FIG. 5 illustrates the equilibration of a sensor response with or without the presence of analyte target such as ATP, Tobramycin, or Cocaine.

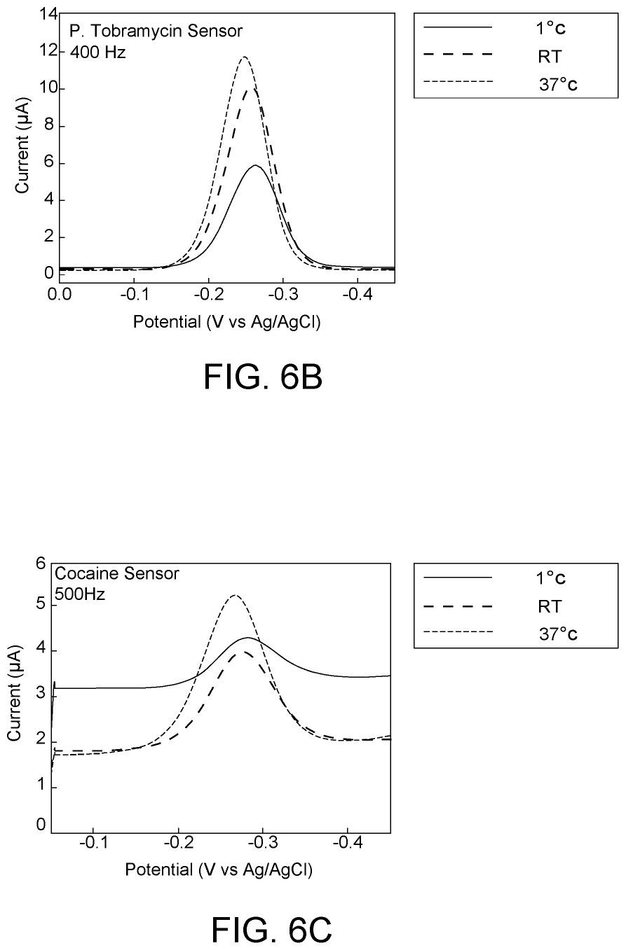

FIGS. 6A-6C are a series of square wave voltammograms (SWV's) of the equilibrations of the Dest. ATP E-AB sensor (FIG. 6A), Parent Tobramycin E-AB sensor (FIG. 6B) and Cocaine E-AB sensor (FIG. 6C). FIG. 6 illustrates the sensors at 1° C., 22° C. and 37° C. The measured current for each of the other sensors change based on the temperature at which they are equilibrated. At lower temperatures (1° C.), the measured current Ip decreases for the three sensors. Without being bound by theory, this could be due to the decreased movement of the oligonucleotides at the surface along with reduced flexibility. Bringing the temperature from 1° C. to a warmer 22° C., an increase of the Ip is observed and finally as the temperature is increased to 37° C. a significant increase in the Ip of the dest. ATP sensor occurs along with a less significant increase in Ip for the parent tobramycin and cocaine E-AB sensors. Without being bound by theory, the increase in Ip could be as a result of increased flexibility of the oligonucleotides as well as their movement at the surface. This potentially results in the significant increase in current for the test. Results for the ATP sensor could be a result of its structure.

### Example 2

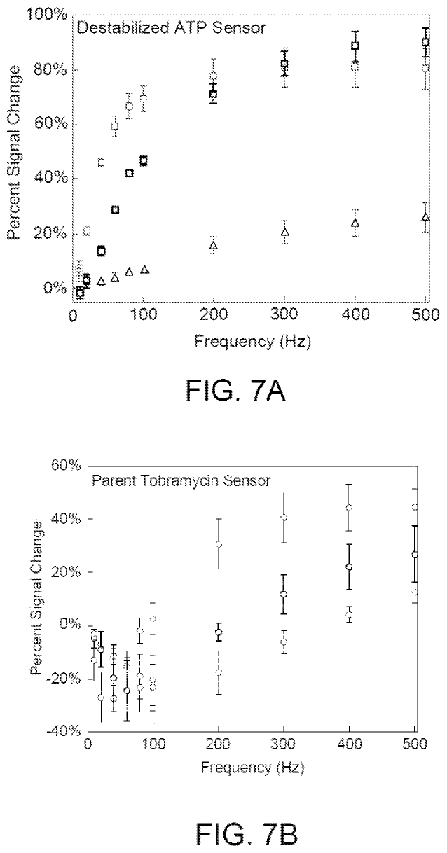

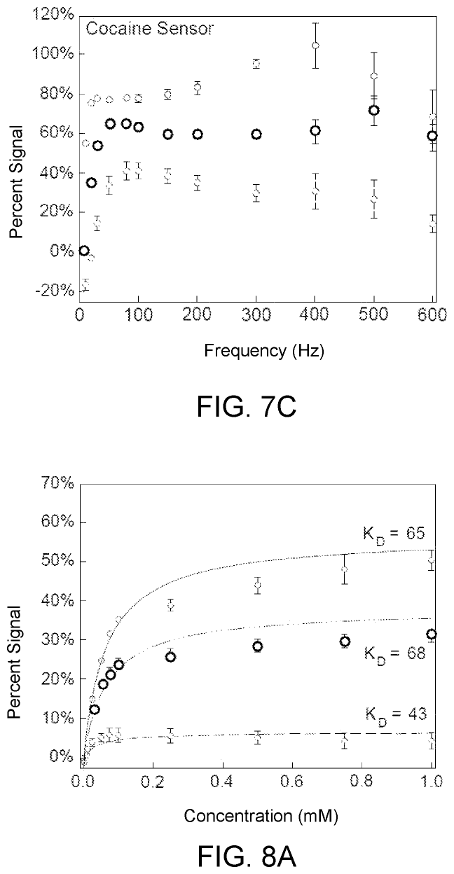

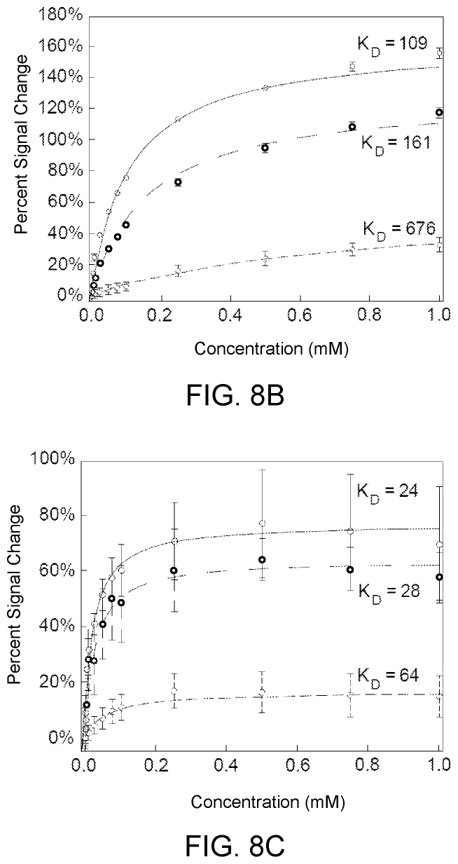

Frequency dependence of E-AB sensors was used as an optimization parameter to better tailor the sensor for an application. Elucidating the frequency dependence based on temperature changes is shown in FIGS. 7 and 8. FIG. 7A is a series of frequency sweeps performed using the Destabilized ATP E-AB sensors at 1° C. (bolded), room temperature (22° C., unbolded) and 37° C. (triangles). FIG. 7B is a series of frequency sweeps performed using the Parent Tobramycin sensors at 1° C. (unbolded), room temperature (22° C., bolded) and 37° C. (dashed lines). FIG. 7C is a series of frequency sweeps performed using the Cocaine sensors at 1° C. (unbolded), room temperature (22° C., bolded) and 37° C. (dashed lines). The frequency sweeps were performed in 20 mM tris buffer (20 mM trizma base, 100 mM NaCl and 5 mM MgCl₂). FIGS. 8A-8C are a series of calibration curves performed using the Parent Tobramycin (FIG. 8A), Destabilized ATP (FIG. 8B) and Cocaine (FIG. 8C) E-AB sensors at 1° C. (unbolded), room temperature (bolded) and 37° C. (dashed lines) at 400, 400 and 500 Hz, respectively.

The aptamers were equilibrated, interrogated, and regenerated in tris buffer at 1° C., 22° C. and 37° C. The temperatures were chosen to cover a range of temperatures in which sensors may commonly be used. Examples of such use include in-vivo, for food testing or for point-of-care testing. The performance of each E-AB sensor was characterized to determine their expected response in room temperature (RT). As shown in FIG. 8, each sensor was equilibrated, interrogated at a saturated concentration of target analyte, and regenerated by rinsing with DI water for 30 seconds. The sensors showed an increase in current that can be quantified and expressed as percent signal change which resulted in a 110%, 38% and 56% increase for the ATP, parent tobramycin and cocaine E-AB sensors, respectively. The sensors were all regenerated back to baseline after rinsing with DI water, which illustrates the advantages of reusability for this class of sensors.

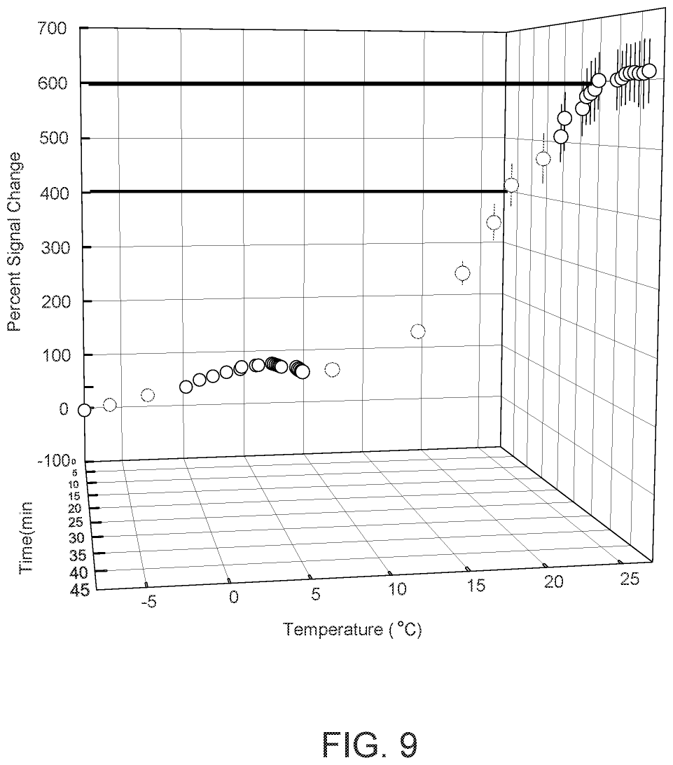

FIG. 9 is a 3-D plot of the destabilized ATP E-AB sensors depicting the increase in percent signal change as a function of time (min) and increasing temperature (° C.) without any target analyte added. The figure shows that as the temperature increases the percent signal increases representing an increase in the peak current (Ip) without the presence of target analyte.

### Materials and Methods

The following chemicals and materials were used as received: Tris-2-carboxyethyl-phosphine (TCEP), Magnesium chloride (MgCl2), sodium chloride (NaCl), Trizma (tris) base (2-amino-2-hydroxymethyl-1,3-propanethiol), sulfuric acid (H₂SO₄), sodium hydroxide (NaOH), 10× Tris-EDTA, and 6-mercapto-1-hexanol (C6-OH), adenosine trisphosphate (ATP), Tobramycin, aminoglycoside antibiotic and norcocaine hydrochloride solution (Sigma-Aldrich, St. Louis, MO, USA) were all used as received. All solutions were prepared using ultrapure water (18.0 MΩ cm at 25° C.) using a Biopak Polisher Millipore ultrapurification system (Millipore, Billerica, MA). The ATP aptamer sequence (5′-CTGGGGGAGTATTGCGGAGGAAA-3′) oligonucleotide sequences were purified using dual HPLC (Thermo-Fisher) and used as received.

### Electrochemical Aptamer-Based (E-AB) Sensor Fabrication on Epoxy-Embedded Electrodes

To fabricate the E-AB sensors, the 2 mm diameter gold disk macro electrodes (CH Instruments, Austin, TX) were hand polished for 2 min in a 1 μm diamond slurry (Buehler, Lake Bluff, IL) followed by polishing for 2 min in 0.05 μm alumina oxide slurry (Buehler, Lake Bluff, IL). All electrodes were rinsed with ultrapure water (Milli-Q Ultrapure Water Purification, Millipore, Billerica, MA, U.S.A.). To remove particulates, the electrodes were sonicated in a low power sonicator for 5 min. Next, the electrodes were electrochemically cleaned in various concentrations of sodium hydroxide and sulfuric acid solutions. Sensor fabrication consists of numerous steps of incubation. First, 2 uL of 100 mM TCEP was added to 1 μL of aptamer and left to incubate for 1 h to perform a disulfide bond reaction. The electrodes were submerged in an aptamer probe solution of 400 nM with 20 mM Trizma Base, 100 mM NaCL and 5 mM MgCl₂ at a pH of 7.45 for 1 h at room temperature. The electrodes were thoroughly rinsed with ultrapure water to rinse excess aptamers. Afterwards the electrodes were submerged in a solution of 30 mM 6-mercapto-1-hexanol for 1.5 h to form the passivation layer. Finally, the electrodes were rinsed with ultrapure water and equilibrated in tris buffer (20 mM Trizma Base, 100 mM NaCl and 5 mM MgCl₂) for 1 hr.

### Electrochemical Measurements

Electrochemical measurements were performed using a CH Instrument 1040C Electrochemical Workstation (CH Instruments, Austin, TX, U.S.A.). Electrochemical measurements were performed in a three-electrode cell consisting of a platinum counter electrode, an Ag/AgCl (3 M KCL) reference electrode (CH Instruments, Austin, TX) and three 2 mm diameter gold disk electrodes as the working electrodes. We used square wave voltammetry (SWV) to interrogate the sensor surfaces. SWV's were collected using frequencies of 400 and 500 Hz with an amplitude of 25 mV, increments potential of 0.001 V, and potential range of −0.05 V to (−0.55 V).

### In-Vivo Testing Setups

In-vivo testing setups used gold-coated acupuncture needles as a working electrode functionalized for aptamer sensor and a gel-pad electrode placed on skin as a reference and counter electrode. In these tests, the sensor is placed in the dermis. Alternately planar gold and gold-disk electrodes functionalized for aptamer sensing sensors were placed ex-vivo and coupled to the dermis via hollow microneedles. These sensors have a temperature dependence that is a mix of skin temperature and environmental temperature, and similarly benefit from temperature correction as taught herein, for example as taught for FIG. 2, where temperature can be determined at or near the aptamer sensor itself.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

What is claimed is:

1. A device for measuring one or more analytes in a sample fluid, the device comprising:
a. at least one aptamer sensor comprising a plurality of aptamers, wherein the sensor provides a measurement of at least one of said analytes and wherein the measurement is affected by changes in temperature (“a temperature-dependent response”);
b. a means to establish fluid communication between the at least one aptamer sensor and the sample fluid;
c. at least one temperature sensor; and
d. a means for correcting a measurement error due to said temperature-dependent response.

2. The device of claim 1 wherein the sample fluid is selected from the group consisting of interstitial fluid, blood and combinations thereof.

3. The device of claim 1 further comprising a reader component with at least one temperature sensor.

4. The device of claim 1 wherein the aptamer sensor is capable of being placed subcutaneously.

5. The device of claim 4 wherein the temperature sensor is capable of being placed subcutaneously.

6. The device of claim 4 wherein the temperature sensor is capable of being placed outside the skin.

7. The device of claim 4 further comprising a first temperature sensor that is capable of being placed subcutaneously and a second temperature sensor that capable of being placed outside the skin.

8. The device of claim 1 wherein the temperature sensor is located no more than about 3 mm from at least one electrochemical aptamer sensor.

9. The device of claim 1 wherein the temperature sensor is located no more than about 1 mm from at least one electrochemical aptamer sensor.

10. The device of claim 1 wherein the temperature sensor is located no more than about 300 μm from at least one electrochemical aptamer sensor.

11. The device of claim 1 wherein the temperature sensor is located no more than about 100 μm from at least one electrochemical aptamer sensor.

12. The device of claim 1 comprising thermal insulation on the device such that the temperature sensor provides adequate prediction of the temperature of at least one electrochemical aptamer sensor.

13. The device of claim 1 further comprising at least one component which is capable of containing a record or prediction of a temperature dependence of the aptamer sensor.

14. The device of claim 13 wherein the aptamer sensor is an electrochemical aptamer sensor with a redox tag attached to each of the plurality of aptamers and said record or prediction additionally comprises a frequency response of the aptamer.

15. The device of claim 13 wherein the aptamer sensor is an electrochemical aptamer sensor with a redox tag attached to each of the plurality of aptamers and said record or prediction additionally comprises an analyte concentration response of the aptamer.

16. The device of claim 13 wherein the aptamer sensor is an electrochemical aptamer sensor with a redox tag attached to each of the plurality of aptamers and said record or prediction additionally comprises a change in sensor response over multiple hours of use of the sensor.

17. The device of claim 1 wherein the aptamer sensor is optical and carries at least one fluorescent tag on each of the plurality of aptamers.

18. A method for measuring one or more analytes in a sample fluid, comprising:
a. exposing a sample fluid comprising one or more analytes to at least one aptamer sensor; the aptamer sensor comprising:
i. a means to establish fluid communication between the at least one aptamer sensor and the sample fluid; and
ii. at least one temperature sensor; and
b. detecting a concentration of said one or more analytes in the sample using the sensor;
wherein the temperature sensor takes at least one measurement and the measurement is a referenced record or prediction of a temperature dependence of the aptamer sensor that predicts change in aptamer sensor response to change in temperature; and
further, the signal measured by the aptamer sensor is adjusted by using the measurement of the temperature sensor and the record or prediction such that the signal from the aptamer sensor accurately represents analyte concentration.

19. The method of claim 18 wherein the sample fluid is selected from the group consisting of interstitial fluid, blood and combinations thereof.

20. The method of claim 18 further comprising a reader component with at least one temperature sensor, and further, wherein the aptamer sensor is disposable after use.

21. The method of claim 18 wherein the aptamer sensor is placed subcutaneously.

22. The method of claim 18 wherein the temperature sensor is placed subcutaneously.

23. The method of claim 18 wherein the temperature sensor is placed outside the skin.

24. The method of claim 18 further comprising a first temperature sensor that is placed subcutaneously and a second temperature sensor that is placed outside the skin.

25. The method of claim 18 wherein the temperature sensor is located no more than about 3 mm from at least one electrochemical aptamer sensor.

26. The method of claim 18 wherein the temperature sensor is located no more than about 1 mm from at least one electrochemical aptamer sensor.

27. The method of claim 18 wherein the temperature sensor is located no more than about 300 μm from at least one electrochemical aptamer sensor.

28. The method of claim 18 wherein the temperature sensor is located no more than about 100 μm from at least one electrochemical aptamer sensor.

29. The method of claim 18 further comprising thermal insulation on the device such that the temperature sensor provides adequate prediction of the temperature of at least one electrochemical aptamer sensor.

30. The method of claim 18 wherein the aptamer sensor is an electrochemical aptamer sensor with a redox tag attached to each of the plurality of aptamers and said record or prediction additionally contains a frequency response of the aptamer.

31. The method of claim 18 wherein the aptamer sensor is an electrochemical aptamer sensor with a redox tag attached to each of the plurality of aptamers and said record or prediction additionally contains an analyte concentration response of the aptamer.

32. The method of claim 18 wherein the aptamer sensor is an electrochemical aptamer sensor with a redox tag attached to each of the plurality of aptamers and said record or prediction additionally contains a change in sensor response over multiple hours of use of the sensor.

33. The method of claim 18 wherein the aptamer sensor is optical and carries at least one fluorescent tag on each of the plurality of aptamers.

34. The method of claim 18 wherein the measurement of the temperature sensor is a direct measure of the temperature at the aptamer sensor.

35. The method of claim 18 wherein the measurement of the temperature sensor is an indirect measure of the temperature at the aptamer sensor.

36. The method of claim 18 wherein the electrochemical aptamer sensor is attached to a user by placing at least a portion of the sensor in the user's skin.

37. The method of claim 18 wherein the temperature sensor is inserted at least 100 μm into a user's skin.

38. The method of claim 18 wherein at least two temperature sensors are placed at different depths into or distances from a user's skin to measure a temperature gradient that predicts temperature of the aptamer sensor.

39. The method of claim 18 wherein the aptamer sensor comprises thermal insulation such that the temperature sensor provides adequate prediction of the temperature of the aptamer sensor.
